(19) United States
(12) Patent Application Publication
(10) Pub. No.: US 2006/0233895 A1
(43) Pub. Date: Oct. 19, 2006

(54) Herbal remedy for treating Lyme disease

(76) Inventor: Paul R. Brown, Madison, CT (US)

Correspondence Address: WIGGIN AND DANA LLP;ATTENTION: PATENT DOCKETING, ONE CENTURY TOWER, P.O. BOX 1832, NEW HAVEN, CT, 06508-1832, US

(21) Appl. No.: 11/401,596

(22) Filed: Apr. 11, 2006

Related U.S. Application Data
(60) Provisional application No. 60/672,118, filed on Apr. 15, 2005.

(51) Int. Cl. A61K 36/539; A61K 36/185; A61K 36/282

(52) U.S. Cl. 424/725; 424/741; 424/777

(57) ABSTRACT

The present invention is directed to a composition for the treatment of Lyme disease, comprising: Uncaria tomentosa (Cat's Claw); Pau d'arco; Scutellaria baicalensis (Baikal Scullcap); Artemisinin; and Sambucus nigra (Elderberry).

### BACKGROUND OF THE INVENTION

1. Field of the Invention

The present invention is directed to remedies for treating Lyme disease, and more particularly to herbal remedies for treating Lyme disease.

2. Brief Description of the Related Art

Lyme disease (Lyme borreliosis) is a bacterial infection spread by certain kinds of ticks. Lyme disease itself is caused by infection with Borrelia burgdorferi (B. burgdorferi) bacteria. In different parts of the United States, different kinds of ticks carry the bacteria that can cause Lyme disease. Deer ticks spread Lyme disease in the northeastern and upper Midwestern United States. Western black-legged ticks spread the disease on the Pacific coast (mostly Northern California and Oregon). The ticks that spread Lyme disease are very small (about the size of a poppy seed or sesame seed), and their bite is usually painless.

Lyme disease develops in three stages. If a person is bitten by a tick carrying Lyme disease bacteria, a rash often develops at the site of the tick bite within 1 to 31 days. The rash (which may look like a bull's-eye) slowly expands and may become very large. Flu-like symptoms may also occur. This early stage of the disease is called early localized Lyme disease. If Lyme disease is not detected and treated properly during the early localized stage, the infection may progress to the second or third stages of Lyme disease and involve the skin, joints, nervous system, and heart.

The second stage of Lyme disease, called early disseminated Lyme disease, may develop several weeks or months after a person becomes infected. It can cause skin problems, joint problems, early nervous system problems, and heart problems. The last stage of the disease, called late persistent Lyme disease, is often the most serious and may develop weeks, months, or, on rare occasion, even years after the initial infection. It can cause joint problems, late nervous system problems, and heart problems.

Lyme disease may be difficult to diagnose because its symptoms are similar to those of many other illnesses. The early, often vague flu-like symptoms can easily be mistaken for another illness, especially when the typical rash of Lyme disease does not occur with them. Later symptoms of untreated Lyme disease, such as joint problems, weakness or numbness in the arms or legs, severe fatigue, or difficulties with memory and thinking, may resemble other forms of arthritis, fibromyalgia, chronic fatigue syndrome, multiple sclerosis, and other conditions.

Lyme disease has been traditionally treated with antibiotics. A recent study found that if a single dose of the antibiotic doxycycline is given within 72 hours after being bitten by an infected tick, the chances of developing Lyme disease can be reduced by as much as 87% (Nadelman RB, Prophylaxis with single-dose doxycycline for the prevention of Lyme disease after an Ixodes scapularis tick bite, New England Journal of Medicine, 345(2), 2001).

A Lyme disease vaccination called LYMErix (SmithKline Beecham) was available for people in high-risk areas. The key ingredient in LYMErix was a genetically engineered protein from the surface of the bacteria B. Burgdorferi that helps stimulate an immune response against the bacteria. The protein, called OspA, stimulates antibodies that disable B. burgdorferi bacteria's ability to infect people. However, OspA triggers autoimmune arthritis in some individuals. Consequently, LYMErix was recently removed from the market.

Treatments for Lyme disease are the subject of several U.S. Patents. U.S. Pat. No. 6,486,130 to Livey, et al. discloses immunogenic formulations comprising different serological forms of OspC to retard or prevent the development of Lyme disease. U.S. Pat. No. 6,368,603 to Jarecki-Black discloses compositions containing a Borrelia burgdorferi antigen that are useful for eliciting an immunological response in a host mammal susceptible to Lyme disease. U.S. Pat. No. 6,303,129 to Alliger, et al. discloses a process for the preparation of a vaccine from substantially viable Borrelia burgdorferi bacteria, and being capable of inducing an immune or therapeutic response against Lyme Disease when administered to a patient.

Recently, the popularity of natural and herbal remedies (hereinafter referred to as “herbal remedies”) has increased among the general public. People are looking to such herbal remedies for the treatment and prevention of a variety of diseases including, but not limited to arthritis, the flu, cancer, heart disease and asthma. Additionally, people use herbal remedies for a variety of reasons, including: avoidance of chemicals found in prescription and over the counter medications; maintenance of a more natural or organic lifestyle; and to avoid overuse of antibiotics.

Many herbs found throughout the world are known for their medicinal properties. One such herb is pau d'arco. Pau d'arco (also known as Ipe Roxo, Ipes, Lapacho, Tabebuia species, Taheebo and Trumpet bush) is a naturally grown herb that is derived from the inner bark of the Tabebuia Avellanedae or Tabebuia Impetiginosa (Taheebo) tree grown in South America. Traditionally, pau d'arco has been used for treatment of fevers, parasitic infections, malaria and dysentery. Additionally, pau d'arco has been shown to inhibit the growth of tumors as well as provide relief and treatment to various skin diseases such as psoriasis. In its native South America, pau d'arco is used topically to treat insect and snake bites as well as other minor skin injuries. Throughout the history of its use, pau d'arco has been shown to have antibacterial properties.

Another herb known for its medicinal properties is Uncaria tomentosa (also known as cat's claw). Uncaria tomentosa is a large woody vine that is found in the Amazon rainforest and other tropical areas of South and Central America. Uncaria tomentosa has been used medicinally for thousands of years to treat gastrointestinal ailments, arthritis, asthma and inflammations. More recently it has been used to treat cancer and AIDS. Generally, Uncaria tomentosa is known for its antibacterial, anti-inflammatory and antioxidant characteristics.

Another herb known for its medicinal properties is Scutellaria baicalensis (also known as Baikal Scullcap, Huang Qin, Hwanggum, Ogon, Scute, and Wogon). The root of this herb has been traditionally used as an antioxidant, an anti-inflammatory, and as a sedative. This herb has also exhibited fever-reducing properties, anti-parasitic properties, and most recently has been used to treat cancer.

Schisandra is a berry producing woody vine that is indigenous to northeastern China and Korea and is known for its medicinal properties. Species of Schisandra include Schisandra chinensis or Schisandra sphenanthera. The fruit of the Schisandra plant has been traditionally used as a liver treatment and to stimulate the spinal cord and brain and to strengthen and quicken reflexes. Alcohol extracts have helped regenerate liver tissue, and have been used clinically in China for infectious hepatitis. Chinese researchers have isolated a number of lignans from the fruit of the Schisandra, some of them showing the ability to lower elevated levels of serum glutamic pyruvic transaminase (SGPT), which is an indicator of hepatitis. The lignans have also shown to act as a central nervous system depressant. Most recently, Chinese herbalists have started to use Schisandra for treating hives and other skin problems such as eczema. Additionally the fruits have been used in prescriptions for coughs, asthma, kidney ailments, chronic diarrhea, neurasthenia, thirst, spontaneous perspiration and night sweating.

Eleutherococcus senticosus (more commonly known as Siberian Ginseng) is an herb found in the Siberian province of Russia. The root has been used medicinally for decades. Generally, Eleutherococcus senticosus is used to reinforce the body's vital energy and to stimulate the immune system. As this herb has been shown to have a beneficial effect on the heart and has shown to increase energy and stamina, it is a popular treatment for depression, fatigue, and reduction of stress.

Artemisinin (also known as Chinese wormwood or Qinghoasu) is a known herbal treatment, and is extracted from the plant Artemesia annua which is found as an annual herb in Asia, most notably in China and Vietnam. Traditionally, this herb has been used to treat parasite infections. Most recently, it has been proven a safe and effective treatment for malaria.

Elderberry (Sambucus nigra, the berry of the black elder tree) is another known herbal treatment, and has been used for bruises, purging, to deter insects, as an eye wash and to treat colds and flu for many years. It is an excellent source of vitamins A, B and C, and its constituents may also strengthen the body's immune system. Elderberries contain bioflavonoids and anthocyanins, which are thought to positively influence cell function.

Due to this renewed interest and popularity of herbal remedies, what is needed is an herbal remedy for the treatment of Lyme disease. The present invention is believed to be an answer to that need.

### SUMMARY OF THE INVENTION

In one aspect, the present invention is directed to a composition for the treatment of Lyme disease, comprising: Uncaria tomentosa (Cat's Claw); Pau d'arco; Scutellaria baicalensis (Baikal Scullcap); Artemisinin; and Sambucus nigra (Elderberry).

In another aspect, the present invention is directed to a method of treating a patient suffering from Lyme disease, comprising the step of administering to said patent a therapeutically effective amount of the above composition.

These and other aspects will become apparent upon reading the following detailed description of the invention.

### DETAILED DESCRIPTION OF THE INVENTION

It has now been found by the present inventor that a composition useful for treating Lyme disease may be prepared by combining Uncaria tomentosa, pau d'arco, Baikal Scullcap, artemisinin, and Sambucus nigra. As described above, each of these ingredients have medicinal and antibacterial properties, and such properties have now been discovered to be useful in treating Lyme disease.

As indicated above, the present invention is a composition for the treatment of Lyme disease, comprising: Uncaria tomentosa (Cat's Claw); Pau d'arco; Scutellaria baicalensis (Baikal Scullcap); Artemisinin; and Sambucus nigra (Elderberry). Each of these ingredients is discussed in more detail below.

Uncaria tomentosa (Cat's Claw) is collected from its large woody vine that is found in the South American rainforest. After collection it is dried, and the herb can be used in a variety of things, such as teas, supplements and topical treatments. Alternatively, the dried herb can be formed into a powdery substance. In the present invention, Uncaria tomentosa is preferably dried and used in the composition of the present invention. It can be purchased commercially and on the internet.

In addition to Uncaria tomentosa, the composition also includes pau d'arco. Pau d'arco is collected from the inner bark of the Taheebo tree found in South America. Typically, only the inner bark is collected, and the tree is not harvested. Once the inner bark is collected, pau d'arco is extracted from the bark and dried. After drying, the herb may be used in a variety of remedies such as supplements and teas. This herb can also be purchased commercially and on the internet.

Scutellaria baicalensis (Baikal Scullcap) is also present in this composition. Typically, the root of Scutellaria baicalensis is harvested and dried. After drying, the root can be ground into a powdered form. The powder can be used in a variety of remedies including supplements, teas, and the like. Additionally, the powder can be mixed with water and used topically to treat skin ailments. This herb can also be purchased commercially and on the internet.

Elderberry (Sambucus nigra, the berry of the black elder tree) is included in the composition of the invention. The flowers, leaves, berries, bark and roots have all been used in traditional folk medicine for centuries. The fruits have been used to make elderberry wine, and when cooked, can be used in pies and jams. The berries contain more vitamin C than any other herb except rosehips and black currant. As mentioned above, elderberry has also been used to treat bruises, purging, to deter insects, as an eye wash and to treat colds and flu for many years. Elderberry is available commercially as a liquid extract or a powder.

Preferably, Uncaria tomentosa, Pau d'arco, Baikal Scullcap, and artemisinin are each present in the composition of the invention in amounts ranging from about 5 to about 30% by weight, more preferably, from between about 8 and 20% by weight, and most preferably from between about 10 and 15% by weight, all based on the total weight of the composition. A particularly preferred amount of each of these ingredients is about 12% by weight, based on the total weight of the composition.

Elderberry (Sambucus nigra) is preferably present in the composition in amounts ranging from about 15 to about 80% by weight, more preferably from about 25 to about 75% by weight, and most preferably from about 40 to about 60% by weight, all based on the total weight of the composition. A particularly preferred amount of elderberry is about 50% by weight, based on the total weight of the composition.

Additional ingredients may also be included in the composition of the present invention. Preferably, one or more of Schisandra chinensis or Schisandra sphenanthera, Eleutherococcus senticosus (Siberian Ginseng), and glutathione are used as additional ingredients in the present preparations. As mentioned above, Schisandra has been used to treat liver disorders, and is available commercially in powder or liquid form. The root of Eleutherococcus senticosus is harvested after the plant has matured to at least two years old. After harvest, the root is typically dried. It can then be used in a variety of medicinal treatments and supplements such as teas, herbal supplement tablets, and the like. The extract of the root can be used in medicinal and herbal supplements. Glutathione is involved in detoxification, and is believed to bind to toxins, such as heavy metals, solvents, and pesticides, and transform them into a form that can be excreted in urine or bile. Glutathione is also an important antioxidant. Preferably, each of these additional ingredients may be present in the composition in amounts ranging from between about 5 and about 15 wt %, based on the total weight of said composition. Most preferably, each of these ingredients is present in the composition at about 8% by weight.

A probiotic may also be included in the composition of the present invention. In general, any probiotic that promotes the growth of microorganisms may be used. Examples of bacteria that may be included in such probiotic include, but are not limited to: L. acidophilus, L. bifidus, L. rhamnosus, L. plantarun, L. salivarius, L. bulharis, L. lactis, L. casei, and L. brevis. Such probiotics are available in liquid and powder form, and are preferably used in the present invention in amounts ranging from between about 0.5 and about 5% by weight, more preferably from about 0.8 and about 2 wt %, and most preferably from about 1.0 and about 1.5 wt %, all based on the total weight of the composition.

The compositions of the present invention may also include a pharmaceutically acceptable carrier. Pharmaceutically acceptable carriers, adjuvants and vehicles that may be used in the compositions of the present invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride (saline), zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The compositions of the present invention may be taken or co-administered with a separate vitamin and/or mineral supplement. An example of such a vitamin and mineral supplement is Centrum®, Centrum® Silver® or other multi-vitamin or multi-mineral supplements that are widely available through pharmacies, drug stores, vitamin stores and the like. While the vitamins and minerals may be combined in one supplement, it is foreseeable that one supplement of only minerals together may be administered with a separate vitamin supplement along with the administration of a composition of the present invention.

The compositions of the present invention may be prepared by combining the desired amounts of the above ingredients to achieve a homogeneous mixture. No particular order of addition of the ingredients or specialized equipment is necessary.

The compositions of the present invention may be administered by any route that effectively treats Lyme disease. Suitable administration methods include, but are not limited to, parenteral methods such as intravenous, subcutaneous and intramuscular and per os (oral, by mouth), or sublingual, and transdermal bolus or continuous infusions of the composition may be used. Oral, sublingual, intravenous, intramuscular, transdermal bolus and continuous infusion methods of administration are preferred.

The compositions of the present invention are preferably administered internally, e.g., orally or intravenously, in the form of conventional pharmaceutical preparations, for example in conventional enteral or parenteral pharmaceutically acceptable excipients containing organic and/or inorganic inert carriers, such as water, gelatin, lactose, starch, magnesium stearate, talc, plant oils, gums, alcohol, Vaseline, or the like. The preparations can be administered in conventional solid forms, for example, tablets, dragees, suppositories, capsules, or the like, or conventional liquid forms, such as suspensions, emulsions, or the like.

If desired, the preparations can be sterilized and/or contain conventional pharmaceutical adjuvants, such as preservatives, stabilizing agents, wetting agents, emulsifying agents, buffers, or salts used for the adjustment of osmotic pressure. The preparations may also contain other therapeutically active materials. The preparation can be made using conventional methods known in the art of pharmaceutical manufacturing.

The preparations of the present invention should include an amount of the compound of the invention effective for treating Lyme disease. The effective amount will depend on the progression and severity of the Lyme disease in a particular patient and is thus within the ordinary skill of the art to determine for any particular host mammal or other host organism. Suitable dosages may be, for example, in the range of about 1 gram to about 200 grams, more preferably from about 3 grams to about 100 grams, and most preferably from about 5 grams to about 60 grams. Preferably, the composition of the invention is administered to a patient from 1-3 times per week, and more preferably 1-2 times per week. The amount and frequency of the administration of the composition of the invention will depend on several variables, and notably on the curative effect of the administered amount. Those skilled in the art of Lyme disease treatment and herbal medicine will be able to determine the proper dosage and frequency of administration for a particular patient.

While the invention has been described above with reference to specific embodiments thereof, it is apparent that many changes, modifications, and variations can be made without departing from the inventive concept disclosed herein. Accordingly, it is intended to embrace all such changes, modifications, and variations that fall within the spirit and broad scope of the appended claims. All patent applications, patents, and other publications cited herein are incorporated by reference in their entireties.

What is claimed is:

1. A composition for the treatment of Lyme disease, comprising:
a. Uncaria tomentosa (Cat's Claw)
b. Pau d'arco;
c. Scutellaria baicalensis (Baikal Scullcap);
d. Artemisinin; and
e. Sarnbucus nigra (Elderberry).

2. The composition of claim 1, wherein each of said Uncaria tomentosa (Cat's Claw), Pau d'arco, Scutellaria baicalensis (Baikal Scullcap), and Artemisinin are present in said composition from between about 5 and about 30 wt %, based on the total weight of said composition.

3. The composition of claim 1, wherein each of said Uncaria tomentosa (Cat's Claw), Pau d'arco, Scutellaria baicalensis (Baikal Scullcap), and Artemisinin are present in said composition from between about 8 and about 20 wt %, based on the total weight of said composition.

4. The composition of claim 1, wherein each of said Uncaria tomentosa (Cat's Claw), Pau d'arco, Scutellaria baicalensis (Baikal Scullcap), and Artemisinin are present in said composition from between about 10 and about 15 wt %, based on the total weight of said composition.

5. The composition of claim 1, wherein said Sambucus nigra (Elderberry) is present in said composition from between about 15 and about 80 wt %, based on the total weight of said composition.

6. The composition of claim 1, wherein said Sambucus nigra (Elderberry) is present in said composition from between about 25 and about 75 wt %, based on the total weight of said composition.

7. The composition of claim 1, wherein said Sambucus nigra (Elderberry) is present in said composition from between about 40 and about 60 wt %, based on the total weight of said composition.

8. The composition of claim 1, further comprising one or more additional ingredients selected from the group consisting of
(1) Schisandra chinensis or Schisandra sphenanthera;
(2) Eleutherococcus senticosus (Siberian Ginseng); and
(3) glutathione.

9. The composition of claim 8, wherein said additional ingredients are each present in said composition from between about 5 and about 15 wt %, based on the total weight of said composition.

10. The composition of claim 1, further comprising a probiotic.

11. The composition of claim 10, wherein said probiotic is present in said composition from between about 0.5 and about 5 wt %, based on the total weight of said composition.

12. The composition of claim 10, wherein said probiotic is present in said composition from between about 0.8 and about 2 wt %, based on the total weight of said composition.

13. The composition of claim 10, wherein said probiotic is present in said composition from between about 1.0 and about 1.5 wt %, based on the total weight of said composition.

14. The composition of claim 1, further comprising a pharmaceutically acceptable carrier.

15. The composition of claim 14, wherein said pharmaceutically acceptable carrier is selected from the group consisting of water, gelatin, lactose, starch, magnesium stearate, talc, plant oils, gums, alcohol, Vaseline, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride (saline), zinc salts, colloidal silica, magnesium trisilicate, polyvinyl pyrrolidone, cellulose-based substances, polyethylene glycol, sodium carboxymethylcellulose, polyacrylates, waxes, polyethylene- polyoxypropylene-block polymers, polyethylene glycol, wool fat, and combinations thereof.

16. A method of treating a patient suffering from Lyme disease, comprising the step of administering to said patent a therapeutically effective amount of the composition of claim 15.

17. The method of claim 16, wherein said therapeutically effective amount ranges from about 1 gram to about 200 grams, more preferably from about 3 grams to about 100 grams, and most preferably from about 5 grams to about 60 grams.

18. The method of claim 16, wherein said administering step takes place from 1-3 times per week, and more preferably 1-2 times per week.

19. The method of claim 16, wherein said administering step is selected from the group consisting of oral, sublingual, intravenous, intramuscular, transdermal bolus and continuous infusion.
